(19) United States
(12) Patent Application Publication
(10) Pub. No.: US 2007/0299292 A1
(43) Pub. Date: Dec. 27, 2007

(54) Paraffin alkylation

(75) Inventors: William M. Cross, Seabrook, TX (US); Lawrence A. Smith, Pasadena, TX (US)

Correspondence Address: OSHA LIANG LLP / CDTECH, 1221 MCKINNEY STREET, SUITE 2800, HOUSTON, TX, 77010, US

(73) Assignee: Catalytic Distillation Technologies

(21) Appl. No.: 11/473,937

(22) Filed: Jun. 23, 2006

(51) Int. Cl. C07C 2/56

(52) U.S. Cl. 585/720; 585/718

(57) ABSTRACT

A liquid acid process is disclosed in which a hydrocarbon component containing an olefin, an olefin precursor or mixture and an isoalkane and a liquid acid catalyst is fed to a downflow reaction zone containing a disperser, under conditions to induce pulse flow at or near the outlet to react the isoalkane and olefin to produce a reaction product and feeding the reaction product to a vaporization zone containing a disperser under conditions to induce pulse flow at or near the outlet of the vaporization zone. A pressure drop across the disperser in the vaporization zone causes partial vaporization of the hydrocarbon which quench es the heat reaction and cooling the unvaporized portion of said reaction product, which is recovered and allowed to separate into an acid phase and hydrocarbon phase containing the alkylate. The acid catalyst and hydrocarbons may be fractally fed to the reaction zone.

This invention was made with Government support under DOE Cooperative Agreement No. DE-FC36-04GO14152 awarded by DOE. The Government has certain rights in this invention.

### BACKGROUND OF THE INVENTION

1. Field of the Invention

The present invention relates to the alkylation of paraffinic hydrocarbon feed stocks. The present invention provides both an improvement in the operating conditions and the feed stock for acid paraffin alkylations. The present invention further provides a process which obtains more efficient mixing of the olefin, paraffin and liquid acid catalyst.

2. Related Information

The common objective of most alkylation processes is to bring isoalkanes (or aromatics) and light olefins into intimate contact with an acid catalyst to produce an alkylation product. In the petroleum refining industry, acid catalyzed alkylation of aliphatic hydrocarbons with olefinic hydrocarbons is a well known process. Alkylation is the reaction of a paraffin, usually isoparaffins, with an olefin in the presence of a strong acid which produces paraffins, e.g., of higher octane number than the starting materials and which boil in range of gasolines. In petroleum refining the reaction is generally the reaction of a C₃ to C₅ olefin with isobutane.

In refining alkylations, hydrofluoric or sulfuric acid catalysts are most widely used under low temperature conditions. Low temperature or cold acid processes are favored because side reactions are minimized. In the traditional process the reaction is carried out in a reactor where the hydrocarbon reactants are dispersed into a continuous acid phase.

Although this process requires the use of strong acid to operate, no other process has been as efficient and it continues to be the major method of alkylation for octane enhancement throughout the world. In view of the fact that the cold acid process will continue to be the process of choice, various proposals have been made to improve and enhance the reaction and, to some extent, moderate the undesirable effects.

U.S. Pat. No. 5,220,095 disclosed the use of particulate polar contact material and fluorinated sulfuric acid for the alkylation. U.S. Pat. Nos. 5,420,093 and 5,444,175 sought to combine the particulate contact material and the catalyst by impregnating a mineral or organic support particulate with sulfuric acid.

Various static systems have been proposed for contacting liquid/liquid reactants, for example, U.S. Pat. Nos. 3,496,996; 3,839,487; 2,091,917; and 2,472,578. However, the most widely used method of mixing catalyst and reactants is the use of various arrangements of blades, paddles, impellers and the like that vigorously agitate and blend the components together, for example, see U.S. Pat. Nos. 3,759,318; 4,075,258; and 5,785,933.

### SUMMARY OF THE INVENTION

There are several aspects to the present invention. The first aspect denotes a process for producing alkylate using sulfuric acid catalyst comprising (a) introducing a hydrocarbon component consisting essentially of an olefin, an olefin precursor or mixture thereof and an isoalkane to a downflow reaction zone containing a disperser,

(b) incorporating a vaporization zone within a vessel containing said reaction zone or in a separate vessel which also contains a disperser, and (c) operating the vaporization zone at the bubble point of said hydrocarbon component to allow vaporization of the hydrocarbon by adjusting the flow ratios of hydrocarbon/acid/vapor within said vaporization zone to operate at or near the pulse flow regime at is outlet.

One embodiment of the first aspect is a process for producing alkylate using sulfuric acid catalyst comprising feeding a hydrocarbon component consisting essentially of an olefin, an olefin precursor or mixture thereof and an isoalkane to a downflow reaction zone containing a disperser, contacting said olefin, an olefin precursor or mixture thereof and said isoalkane in the presence of liquid sulfuric acid catalyst and react under conditions of temperature and pressure whereby the heat of reaction is at the bubble point of the hydrocarbon component thereby producing vapor, said vapor inducing near pulse flow or pulse flow at or near an outlet to produce a reaction product, and feeding the reaction product to a vaporization zone containing a disperser under conditions to induce near pulse flow or pulse flow at or near an outlet of the vaporization zone wherein a pressure drop across the disperser causes partial vaporization of the hydrocarbon component of said reaction product, quenching of the heat of reaction and cooling of an unvaporized portion of said reaction product.

The second aspect of the present invention focuses on the use of fractal scaling to feed the three components into the reactor in a process for the alkylation of an olefin with and alkane in the presence of a liquid acid catalyst comprising the steps of: fractally feeding a liquid acid catalyst through a fractal distributor to distribute the liquid acid catalyst evenly; fractally feeding a hydrocarbon comprising an isoalkane and an olefin through said fractal distributor to distribute the hydrocarbon evenly, preferably in a downflow reaction zone containing a disperser, under conditions to induce pulse flow at or near the outlet, to produce a reaction mixture thereof with said acid catalyst; reacting said isoalkane and said olefin to produce an alkylate; recovering a reaction product comprising said reaction mixture and said alkylate; and separating said reaction product into a hydrocarbon phase and an aqueous phase.

### BRIEF DESCRIPTION OF THE DRAWING

FIG. 1 is a top plan view of an acid predistributor plate of the preferred fractal distributor.

FIG. 2 is a bottom plan view of an acid predistributor plate of the preferred fractal distributor.

FIG. 3 is a bottom plan view of an acid predistributor plate of the preferred fractal distributor with the insert removed and showing the flow channels.

FIG. 4 is a top plan view of a final acid distribution plate of the preferred fractal distributor.

FIG. 5 is a bottom plan view of a final acid distribution plate of the preferred fractal distributor.

FIG. 6 is a bottom plan view of a final acid distribution plate of the preferred fractal distributor with an insert removed to shown the flow channels.

FIG. 7 is a bottom plan view of a hydrocarbon distribution plate of the preferred fractal distributor.

FIG. 8 is a top plan view of a hydrocarbon distribution plate of the preferred fractal distributor.

FIG. 9 is a top plan view of a hydrocarbon distribution plate of the preferred fractal distributor win the insert removed to show the flow channels.

FIG. 10 is a top plan view of a plate assembly of the preferred fractal distributor comprising the acid predistributor plate, final acid distributor plate and hydrocarbon distributor plate.

FIG. 11 is a bottom plan view of a plate assembly of the preferred fractal distributor comprising the acid predistributor plate, final acid distributor plate and hydrocarbon distributor plate.

FIG. 12 is a top plan view of the initial piping for one phase of the preferred fractal distributor.

FIG. 13 is a top plan view of the final piping for one phase of the preferred fractal distributor.

FIG. 14 is a top plan view of one section showing the final piping for two phases of the preferred fractal distributor.

FIG. 15 is a schematic representation of the first aspect of the present apparatus in which the present alkylation process may be carried out.

### DETAILED DESCRIPTION OF THE INVENTION

The present invention offers an improved mixing of the reactants and the catalyst and process for producing and separating an alkylate product using liquid acid as catalyst.

The present process preferably employs two downflow zones packed with contacting internals or packing material (which may be inert or catalytic) through which passes a concurrent multi phase mixture of sulfuric acid, hydrocarbon solvent and reactants at the boiling point of the system.

In the first zone the system comprises a hydrocarbon phase and an acid/hydrocarbon emulsion phase. A significant amount of sulfuric acid is held up on the packing. Reaction takes place between the descending hydrocarbon phase and the sulfuric acid dispersed on the packing. Olefin continuously dissolves into the acid phase and alkylate product is continuously extracted into the hydrocarbon phase. The first zone is preferentially operated liquid. The pressure is preferentially higher at the top of the first zone than at the bottom.

After the liquid continuous zone the mixture moves t a second zone which is operated either liquid or vapor continuous. Vapor is created in this zone by adjusting the pressure and hydrocarbon composition to control the boiling point temperature. Vapor is created in this zone by adjusting the pressure and hydrocarbon composition to control the boiling point temperature. The rate of vaporization is a function of the heat of the overall reaction: olefin+isoparaffin→alkylate. Pulse flow is obtained at high gas and liquid flow rates. The pulse flow or transitional flow obtained in this second zone is obtained at high gas and liquid flow rates. The pulses are characterized by large mass and heat transfer rates. Increased catalyst wetting and a continuous mixing between parallel flowing rivulets diminish flow maldistribution. In addition, the formation of local hot spots is reduced, leading to an intrinsically safer process and diminished catalyst deactivation. The pulses continuously mobilize the stagnant liquid holdup to the point where its stagnant nature disappears. Since stagnant holdup represents about 10 to 30 percent of the total liquid holdup in trickle flow operations, its more dynamic character during pulsing flow enhances reactor performance. Axial dispersion is considerably less compared to trickle flow, due to effective radial mixing between the different parallel flowing liquid streams and disappearance of stagnant liquid hold up. Especially undesired consecutive reactions are reduced to lower levels due to better overall plug flow behavior. A further advantage of pulsing flow is much higher radial conductivity. In some cases, depending on the pulse frequency, significant changes in both yield and selectivity occur.

In this embodiment the process for the alkylation of an olefin with and alkane in the presence of a liquid acid catalyst comprises the steps of:

(a) feeding to a reaction zone having an inlet and an outlet and containing a disperser, a liquid acid catalyst and a hydrocarbon comprising isoalkane and an olefin;

said disperser intimately contacting the liquid acid catalyst, the isoalkane and the olefin to react a portion of the isoalkane with the olefin to produce a reaction mixture containing liquid acid catalyst, hydrocarbon comprising unreacted isoalkane, unreacted olefin and alkylate product;

(b) feeding the reaction mixture to a reaction/vaporization zone having an inlet and an outlet containing and a disperser under conditions to vaporize portion of the hydrocarbon to produce a vapor and cool the reaction mixture and to induce a flow regime near a pulse flow regime at the outlet to produce a stable and tight emulsion;

(c) withdrawing from the vaporization zone a vapor phase containing unreacted isoalkane, unreacted olefin and alkylate and a liquid phase containing liquid acid catalyst and alkylate product; and

(d) separating the liquid acid catalyst from the alkylate product.

The main benefit with pulse regime reactor operation is that of increased mass transfer and heat transfer due to the associated turbulence produced. When the catalyst physical characteristics are optimized and the reaction kinetics are not limiting, increasing mass transfer is a key to increasing the process performance.

The pulse may be induced by increasing the gas rate while maintaining the liquid rate until a pressure drop sufficient to induce the pulse flow is achieved. Further, the pulsing may be dampened while keeping the mixing characteristics by utilizing a second liquid of different viscosity. The dampening reduces the wear and tear on catalysts and also maintains more even flow rates.

In the vaporization zone the combination of the heat of reaction and the pressure drop across the disperser causes vaporization of part of the hydrocarbon.

Adjusting the flow rates and the degree of vaporization, controls the pressure drop across the vaporization zone. The type of packing also influences the pressure drop due to the acid phase hold-up. The product mixture before fractionation is the preferred circulating solvent. The acid emulsion separates rapidly from the hydrocarbon liquid after exiting the vaporization zone and is normally recycled within only a few minutes residence time in the bottom phase separator. Because the products are in essence rapidly extracted from the acid phase (emulsion), the reaction and/or emulsion promoters used in conventional sulfuric acid alkylation processes may be added without the usual concern for breaking the emulsion. The process may be described as hydrocarbon continuous as opposed to acid continuous.

Preferably, the disperser comprises a conventional liquid-liquid coalescer of a type which is operative for coalescing vaporized liquids. These are commonly known as “mist eliminators” or “demisters”, however, in the present invention the element functions to disperse the fluid materials in the reactor for better contact. A suitable disperser comprises a mesh such as a co-knit wire and fiberglass mesh. For example, it has been found that a 90 needle tubular co-knit mesh of wire and multi-filament fiberglass such as manufactured by Amistco Separation Products, Inc. of Alvin, Tex., can be effectively utilized, however, it will be understood that various other materials such as co-knit wire and multi filament teflon (Dupont™), steel wool, polypropylene, PVDF, polyester or various other co-knit materials can also be effectively utilized in the apparatus. Various wire screen type packings may be employed where the screens are woven rather than knitted. Other acceptable dispersers include perforated sheets and expanded metals, open flow cross channel structures which are co-woven with fiberglass or other materials such as polymers co-knit with the wire mesh expanded or perforated sheets. Additionally the multi-filament component may be catalytic. The multi-filament catalytic material may be polymers, such as sulfonated vinyl resin (e.g., Amberlyst) and catalytic metals such as Ni, Pt, Co, Mo, Ag.

The disperser comprises at least 50 volume % open space up to about 97 volume % open space. Dispersers are position within the first zone. Thus, for example, the multi filament component and the structural element, e.g., knit wire, should comprise about 3 volume % to about 50 volume % of the total disperser, the remainder being open space.

Suitable dispersers include structured catalytic distillation packings which are intended to hold particulate catalysts, or structured distillation packings composed of a catalytically active material, such as that disclosed in U.S. Pat. No. 5,730,843 which is incorporated herein in its entirety and which discloses structures that have a rigid frame made of two substantially vertical duplicate grids spaced apart and held rigid by a plurality of substantially horizontal rigid members and a plurality of substantially horizontal wire mesh tubes mounted to the grids to form a plurality of fluid pathways among the tubes, said tubes being empty or containing catalytic or non catalytic materials; and structured packings which are catalytically inert which are typically constructed of corrugated metal bent at various angles, wire mesh which is crimped, or grids which are horizontally stacked one on top of the other, such as disclosed in U.S. Pat. No. 6,000,685 which is incorporated herein in its entirety and which discloses contact structures comprising a plurality of sheets of wire mesh formed into vee shaped corrugations having flats between the vees, said plurality of sheets being of substantially uniform size having the peaks oriented in the same direction and substantially in alignment, said sheets being separated by a plurality of rigid members oriented normally to and said resting upon said vees.

Other suitable dispersers include: (A) random or dumped distillation packings which are: catalytically inert dumped packings contain higher void fraction and maintain a relatively large surface area, such as, Berl Saddles (Ceramic), Raschig Rings (Ceramic), Raschig Rings (Steel), Pall rings (Metal), Pall rings (Plastic, e.g. polypropylene) and the like and catalytically active random packings which contain at least one catalytically active ingredient, such as Ag, Rh, Pd, Ni, Cr, Cu, Zn, Pt, Tu, Ru, Co, Ti, Au, Mo, V, and Fe as well as impregnated components such a metal-chelate complexes, acids such as phosphoric acid, or bonded, inorganic, powdered materials with catalytic activity; and (B) monoliths which are catalytically inert or active which are structures containing multiple, independent, vertical channels and may be constructed of various materials such as plastic, ceramic, or metals, in which the channels are typically square; however, other geometries could be utilized, being used as such are coated with catalytic materials.

In addition to using a disperser to feed the mixture, a single fractal distributor which distributes two fluids independently up until they are combined at the final outlet, such as disclosed in U.S. Pat. No. 6,742,924, may be used in the reactors for the initial distribution of light and heavy (liquid acid catalyst) into the alkylation reactor. A single fractal distributor achieves distribution of two fluids independently up until they are combined at the final outlet by providing independent fractal flow channels up until reaching the final drip points of the last layer of fractal plates. The main problem associated with the system is that the overhead piping for the two separate fluids may interfere with the final drip pattern. Preferably, in the system, one of the fluids enters the final fractal plate from below. Interference with the piping is achieved by offsetting, for example by rotating, the second fluid distribution header such that the downward piping passes between the fractal plates. A mathematical formula for the degree of offset from the radius is based upon the circumference and number of plates.

“Fractal scaling,” as contemplated herein, is a recursive process by which an algorithm is applied in successive stages, each time to process the outputs from an immediately preceding stage. A simple case for purposes of illustration is to apply the algorithm to “divide a flow stream into two equal flow streams.” According to this example, a flowing stream is divided into two equal streams of half the initial volume during a first stage. Each of the two resulting streams is then similarly divided to produce a total of four equal streams of reduced volume in a second stage. Those four resulting streams are then divided into eight equal streams of reduced volume in a third stage, and soon, through as many stages as are desired to achieve the distribution of fluid flow required for a particular application.

Mathematical models of fractal geometry assume that each division at each stage is identical and that precisely identical geometry is followed through each branch of successive stages. In practice, it is recognized that absolute adherence to a mathematical model is impractical. Accordingly, fractal devices are usually constructed to approximate a theoretical model. That is, because of manufacturing and space constraints, commercial fractals often make use of “similar”, rather than “identical” fractal patterns. The practical consequences of this departure from theoretical are generally minimal within the practical realm.

Fractals may be constructed as an entire device, or multistage segment of such a device, as a unitary structure, e.g., through investment, shell or lost wax casting techniques. Multilevel fractals are more conveniently provided, however, through the use of a stack of fractal elements in an assembly, or “fractal stack.” To avoid redundancy of description, this disclosure gives primary emphasis to fractal stacks utilized as distributors.

The individual elements of a typical fractal stack are three-dimensional components, structured and arranged for juxtaposed assembly in a specified sequence. Each fractal element is provided with channels and ports constituting a portion of a fractal fluid scaling array. Various portions of the scaling array may be assigned to individual elements, those portions being selected such that a practical recursive fractal array results from the assembly of the elements, in proper sequence, into the fractal stack. A presently preferred arrangement assigns the fluid flow channels of a specified fractal stage to a single specified fractal element. It is within contemplation to assign channels of different fractal stages to a single fractal element, and it is also within contemplation to divide channels of a specified fractal stage among a plurality of fractal elements. The channels associated with a particular element may be positioned on a single side or on the opposed sides. In the latter case, the channels of a fractal stage may be defined by juxtaposed matching grooves at the interfaces between adjacent elements.

An exemplary fractal element has a relatively large cross section normal the direction of fluid flow to accommodate the largest fractal pattern in the stack. This pattern is typically that of the final fractal stage, and its “footprint” is dependent upon (among other things) the fractal number (the number of stages) accommodated by the stack. A relatively small height dimension is required to accommodate flow channels arranged in a fractal pattern within, (most often openly communicating with either or both interfacing surfaces of the element). Such elements take the form of short prisms, usually cylindrical and are designated “fractal plates,” for purposes of this disclosure and may be arranged in a cylindrical vessel for use. Fractal plates may be stacked upon one another such that fractal distribution to progressively smaller scales occurs as fluid passes through the stack. The device therefore acts as a fluid distributor. Near limitless scaling of fluid motion can be accomplished with this invention by the addition of fractal plates to the stack, that is, by increasing the fractal number of the stack.

Most often, portions of the fractal pattern are provided on structural elements assembled in stacked arrangement with respect to each other. The structural elements are typically, approximately congruent geometric solids with flow channels arranged therein. The invention is thus applied in practice to a fractal fluid system in which recursive flow paths are arranged in a fractal pattern including generations of progressively increasing or decreasing scale. The improvement of the invention generally comprises providing portions of the fractal pattern in stacked arrangement with respect to each other, whereby to avoid intersection of recursive flow channels. The generations of progressively increasing or decreasing scale are typically positioned between an inlet and an outlet, whereby to modify the scale of fluid flow through the system. As used in the present invention successively arranges such generations of structural flow channels at different distances from the inlet in the direction of the outlet in conformance to the fractal pattern so as to constitute fractal elements. Ideally, these fractal elements comprise plates which contain fractal patterns, one stacked upon another, to provide a fractal stack constituting a means for fluid distribution at progressively different scales as fluid passes through the stack from its inlet to its outlet. The inlet may be located to direct fluid to either the largest or smallest scale fractal generation.

Particularly when the stack is operated as a distributor, it may include a finishing structure at one (outlet) end, structured and arranged to promote even distribution of fluid normal to the direction of fluid flow through the stack. The finishing structure is preferably constructed and arranged to provide multiple channel tortuous pathways for fluid exiting the fractal pattern. The opposite (inlet) end of the stack may comprise a structural element containing distribution channels arranged to receive fluid from a primary inlet and to distribute scaled quantities of that fluid to respective inlets of a first generation of the fractal pattern.

Because fractals are, by definition, invariant to scaling, when employed in the present process they can be used for any size application and still provide any desired range of fluid scaling. Fractal devices theoretically enables infinite scaling of fluids.

The hydrocarbon feedstock undergoing alkylation by the method of the present invention is provided to the reaction zone in a continuous hydrocarbon phase containing effective amounts of olefinic and isoparaffinic starting materials which are sufficient for forming an alkylate product. The olefin:isoparaffin mole ratio in the total reactor feed should range from about 1:1.5 to about 1:30, and preferably from about 1:5 to about 1:15. Lower olefin:isoparaffin ratios may also be used.

The olefin, preferably aliphatic, component should preferably contain 2 to 16 carbon atoms and the isoparaffin component should preferably contain 4 to 12 carbon atoms. Representative examples of suitable isoparaffins include isobutane, isopentane, 3-methylhexane, 2-methylhexane, 2,3-dimethylbutane and 2,4-dimethylhexane. Representative examples of suitable olefins include butene-2, isobutylene, butene-1, propylene, pentenes, ethylene, hexene, octene, and heptene, merely to name a few. In lieu of feeding the olefins the oligomer of the olefin may be fed as described in U.S. Pat. No. 6,995,296. The great advantage of using the oligomer is that although acid alkylations are extremely exothermic and require substantial refrigeration to maintain the temperature in the optimum range to prevent side reactions, the reaction of oligomers with the isoalkanes to produce the alkylate in the same yields requires less refrigeration making the process less expensive for the same yield of useful product.

In the fluid process the system uses hydrofluoric or sulfuric acid catalysts under relatively low temperature conditions. For example, the sulfuric acid alkylation reaction is particularly sensitive to temperature with low temperatures being favored in order to minimize the side reaction of olefin polymerization. Petroleum refinery technology favors alkylation over polymerization because larger quantities of higher octane products can be produced per available light chain olefins. Acid strength in these liquid acid catalyzed alkylation processes is preferably maintained at 88 to 94% by weight using the continuous addition of fresh acid and the continuous withdrawal of spent acid. Other acids such as solid phosphoric acid may be used by supporting the catalysts within or on the packing material.

Preferably, the process of the present invention should incorporate relative amounts of acid and hydrocarbon fed to the top of the reactor in a volumetric ratio ranging from about 0.01:1 to about 2:1, and more preferably in a ratio ranging from about 0.05:1 to about 0.5:1. In the most preferred embodiment of the present invention, the ratio of acid to hydrocarbon should range from about 0.1:1 to about 0.3:1. The feed of the liquid acid to the top of the reactor maybe accomplished through a fractal distribution system as heretofore described designed with sufficient fractal stages to give even distribution over the entire cross sectional area of the reactor. Such feeding is known herein as fractally feeding the liquid acid. The liquid hydrocarbons are fed together into the last fractal stage prior to entering the reactor.

Additionally, the dispersion of the acid into the reaction zone should occur while maintaining the reactor vessel at a temperature ranging from about 0° F. to about 200° F., and more preferably from about 35° F. to about 130° F. Similarly, the pressure of the top of the reactor vessel should be maintained at a level ranging from about 0.5 ATM to about 50 ATM, and more preferably from about 0.5 ATM to about 20 ATM. Most preferably, the reactor temperature should be maintained within a range from about 15° F. to about 110° F. and the reactor pressure should be maintained within a range from about 0.5 ATM to about 5 ATM.

In general, the particular operating conditions used in the process of the present invention will depend to some degree upon the specific alkylation reaction being performed. Process conditions such as temperature, pressure and space velocity as well as the molar ratio of the reactants will affect the characteristics of the resulting alkylate product and may be adjusted in accordance with parameters known to those skilled in the art.

An advantage of operating at the boiling point of the present reaction system is that there is some evaporation which aids in dissipating the heat of reaction and making the temperature of the incoming materials closer to that of the materials leaving the reactor as in an isothermal reaction.

Once the alkylation reaction has gone to completion, the reaction mixture is transferred to the vaporization zone from which hydrocarbon vapor is removed and the remaining acid hydrocarbon removed to a suitable separation vessel where the hydrocarbon phase containing the alkylate product and any unreacted reactants is separated from the acid. Since the typical density for the hydrocarbon phase ranges from about 0.6 g/cc to about 0.8 g/cc and since densities for the acid generally fall within the ranges of about 0.9 g/cc to about 2.0 g/cc, the two phases are readily separable by conventional gravity settlers. Suitable gravitational separators include decanters. Hydrocyclones, which separate by density difference, are also suitable.

One alkylation embodiment is shown in the FIG. 15 which is a simplified schematic representation of the apparatus and flow of the process. Such items as valves, reboilers, pumps, etc., have been omitted.

Fresh makeup sulfuric acid is fed via flow line 101 and combined with recycle acid in flow line 102 in flow line 104 and fed using a fractal distribution system (fractally feeding) into a first reactor 10 containing a disperser 12. Isobutane and olefin are fed via flow line 105 into the last stage of the fractal distributor with the combined acid hydrocarbons distributed over the cross sectional area of the reactor 10. A recycle hydrocarbon stream in flow line 106 is also fed into the last stage of the fractal distributor. Nominally, the preferred operation for the reactor 10 using sulfuric acid is: temperature 15-70 F; pressure drop 0.5-10 psi/ft of packing height; disperser void fraction 0.8-0.99; % volume acid entering reactor 30% or greater; % volume acid in the pack zone 30% or greater. As contact is made reaction occurs, creating heat, and the reaction mixture increases in temperature. Recycle rates of hydrocarbon may be adjusted to maintain a particular temperature rise. Nominally the temperature rise across this reactor 10 is maintained to less than 5 F, although a higher temperature could be acceptable.

The effluent mixture is withdrawn from the reactor 10 via flow line 109 and fed to a vessel 20 also containing a disperser 22. Hydrocarbon recycle is added at the inlet via flow line 107 and line 110. In reactor 10 the effluent is allowed to vaporize. At the inlet of reactor 20 both a liquid hydrocarbon phase and an acid catalyst phase exist and the inlet pressure of the reactor 20 is at or very near the bubble point of the hydrocarbon phase. As the flowing hydrocarbon passes the zone it flashes due to pressure drop across the disperser which quenches the heat of reaction from reactor 10, thus cooling the composite acid and hydrocarbon stream exiting the reactor 20.

For a fixed disperser (same disperser used in both reactor 10 and 20) the pressure drop across the reactor 20 is higher than that of reactor 10 due to the presence of the vapor. Nominally the preferred % volume of acid (on a total liquid basis—no vapor) is held to 30% or greater in reactor 20. Additionally, mass fluxes utilized near and/or at the exit of reactor 20 are within what is considered the “transition” region of the “pulse flow” region of a two phase hydraulic map. Transitional flow represents a narrow region of mass fluxes between trickle flow and pulse flow. This region is essentially on the transition line of the flow map separating pulse flow from trickle flow, which lies at a point where a small change in liquid flow causes a relatively large change in differential pressure drop across the bed. A more complete discussion and description of the “transition” and “pulse flow” regimes is contained in U.S. Pat. No. 6,774,275 which is incorporated herein by reference. The operational window is desired in order top provide desirable high heat and mass transfer rates. A second benefit of operating within this regime is that it produces a more stable acid and “tighter” emulsion (as measured by the time for settling) than operation outside this region. Emulsion tightness is herein indicated by a density range of the emulsion upon a settling time of between 30 seconds to 2 minutes. This density is between 1.2 and 1.7 g/cc and nominal targets are typically for a range of between 1.3-1.25 g/cc. For sulfuric acid alkylation, formation of a more stable acid emulsion containing small hydrocarbon drops aids product quality and overall unit performance. Since general mixing of sulfuric acid and hydrocarbon is somewhat difficult due to the large density difference between the fluids and their associated interfacial tension, it nominally requires a certain minimum horse power per barrel per day alkylate rate(hp/bbl/day) to achieve. In the past (U.S. Par. No. 3,155,742) this value has been indicated to be in the range of 0.1-0.15 hp/bbl/day. Utilizing the herein disclosed flow regime to establish the desired emulsion can reduce this overall minimum energy requirement down to values of 0.03-0.05 hp/bbl/day (reported here as energy required reactors 10 and 20).

The effluent mixture is withdrawn from reactor 20 via flow line 111 and the vapor removed via flow line 112. The liquid is taken by flow line 113 to settler/coalescer 30 wherein the liquid hydrocarbon phase is separated from the sulfuric acid phase. A hydrocarbon stream 114 is removed and sent to a distillation column (not shown) for separation of the alkylate from the iC₄/olefin. The alkylate is removed as product while the iC₄/olefin is recycled to reactor 10 or 20 (not shown).

A recycle stream is also removed from the settler/coalescer via flow line 108 with a portion being recycled to reactor 10 via flow line 106 and a portion recycled to reactor 20 via flow line 107. Acid is removed from the settler/coalescer via flow line 102 with a portion being sent to spend acid storage via flow line 102 with the remainder being recycled back to reactor 10.

The preferred distributor described herein stems from the same basis as U.S. Pat. No. 6,616,327 which incorporated by reference herein in its entirety, in which fractal patterns are utilized. To increase the number of distribution points per square foot of distributor, the fractal pattern is repeated on the next layer of distribution. Each layer of distribution is typically a formed, shaped or cut out plate. For a fractal distributor, in which each fractal plate contains enough fractal elements to expand its number of inlets, each into 6 fractal branches, feeding 6 separate outlets and maintains the same geometric arrangement on a plate by plate basis, the number of distribution drip points goes up as nm where n is the number of fundamental fractal divisions per plate and n^{m} is the number of plates, such that four fractal plates provide a total of 1296 drip points.

For planar geometry the smallest building block of the fractals stems from linear branching starting at the node located in the centroid of the overall shape. The shortest path length from the starting node to the outlet nodes is a straight line.

When these fundamental building blocks are combined to form more complex fractal distributor (with the restriction that all outlet nodes are equidistant apart) the paths between the central node (or centroid of the overall shape) to the outlet nodes becomes more complex in order to provide the same path length or fractal geometry. This particular fractal patterning is more fully described in U.S. Pat. No. 6,616,327, previously incorporated by reference.

Although is it recognized that to hold to an exact fractal pattern is not practical, the point in striving for this geometric arrangement is to obtain the same flow path length to each drip point. This allows for a robust distributor design as every flow path is hydraulically equivalent. From a distribution standpoint this allows for large variation in overall flowrates and the capability to handle changes in fluid properties (such as viscosity) while maintaining equid distribution per point.

The object is to provide a single fractal distributor which distributes two fluids independently up until they are combined at the final outlets. This is allowed by providing independent fractal flow channels up until reaching the final drip points of the last layer of fractal plates.

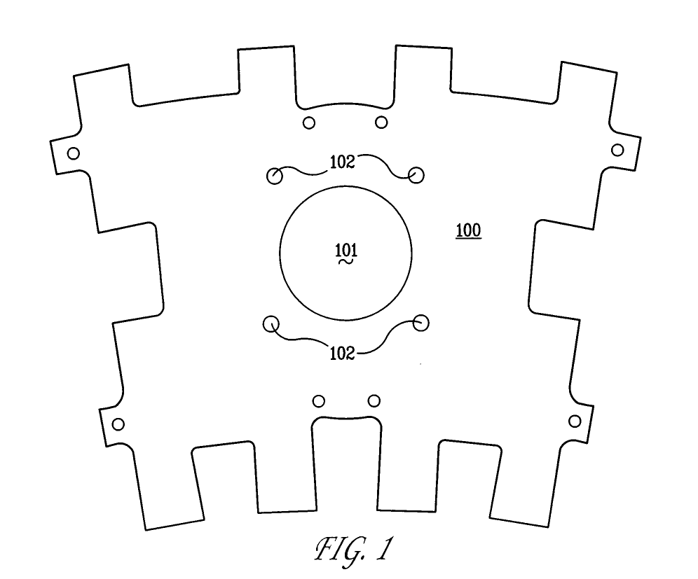

For general construction of fractal plates the reader is referred to U.S. Pat. No. 6,616,327. One section of fractal plates shaped as a pie wedge is shown in FIGS. 1-12. The particular fractal plates have been designed such that the problem of interference between inlet piping for two phases is minimized. The first phase in the illustrated case is a viscous fluid, sulfuric acid, and the second phase is a hydrocarbon phase, comprising isobutane and butylenes. The final mixing occurs in the last plate, wherein the sulfuric acid enters from the top and the hydrocarbon enters from the bottom.

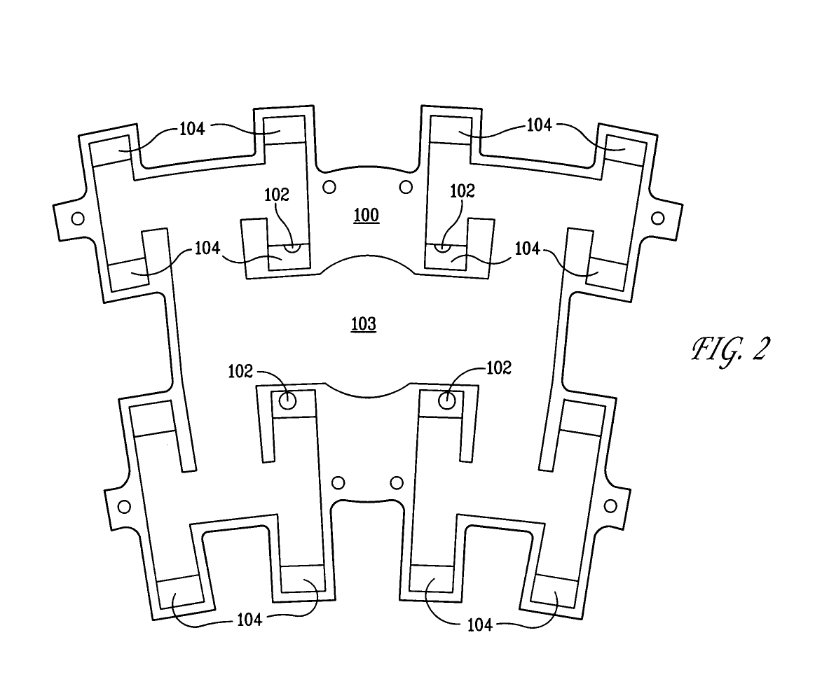

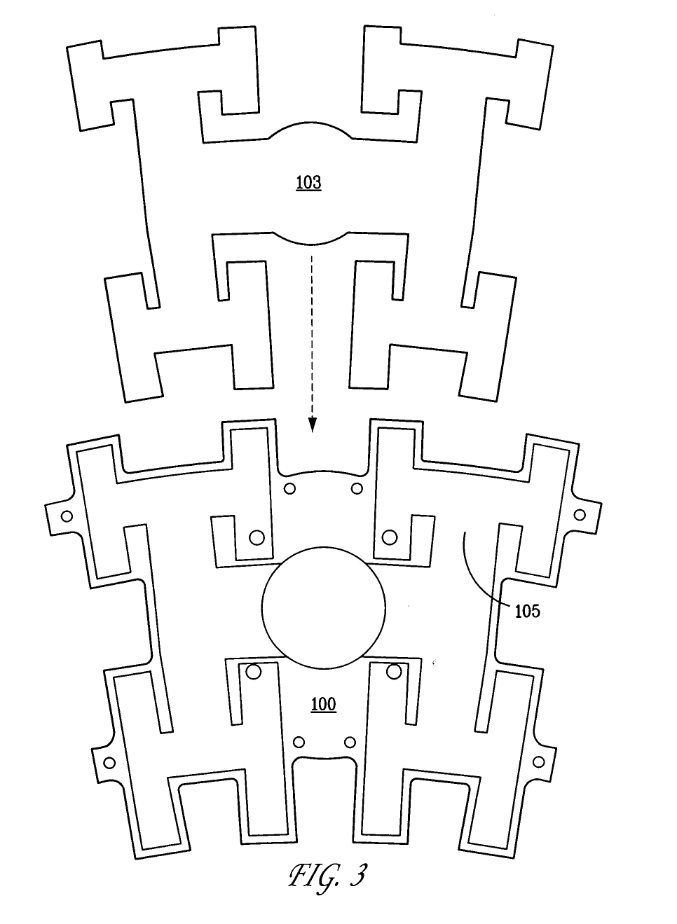

Referring now to the figures, a preferred embodiment includes three plates: 1) an acid (or highly viscous fluid) predistribution plate; 2) a final acid distribution plate and 3) a hydrocarbon distribution plate. Both feeds enter the vessel from above and then must be connected to their respective inlets. In FIG. 1 the acid predistribution plate 100 is shown from the top. The acid inlet is shown at 101. The holes 102 are for bolts that hold the plates together. FIG. 2 shows the acid predistribution 100 plate from the bottom. Insert 103 covers the flow channels from the inlet to the initial drip points 104. In FIG. 3, the insert 103 has been removed exposing the flow channels 105 and the inlet 101.

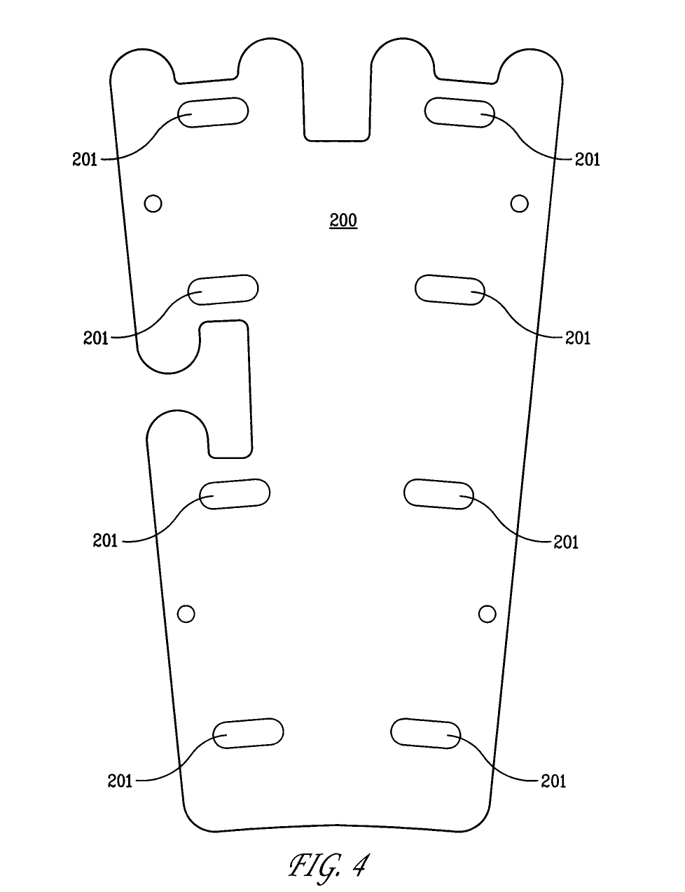

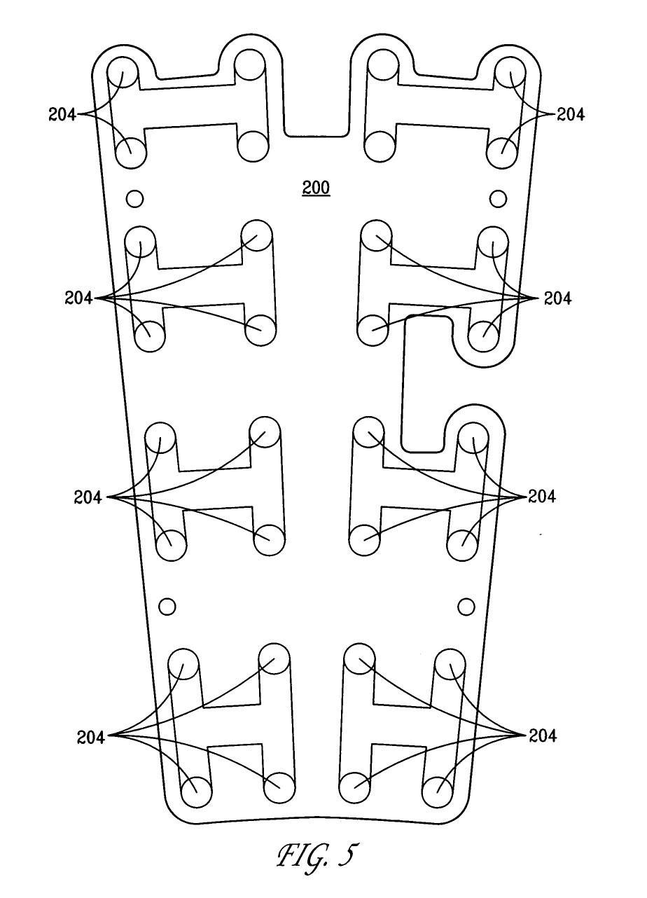

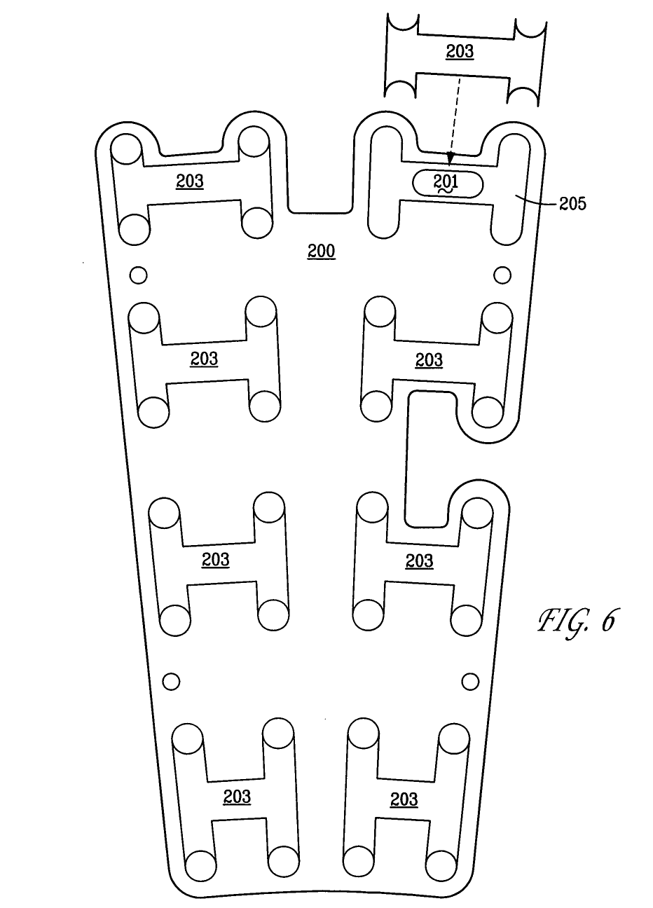

Referring now to FIGS. 4-6, the final acid distribution plate 200 is shown. In use, there are two final acid distribution plates 200 for each acid predistribution plate 100. Each final acid distribution plate has eight inlets 201, which match up to each of the initial drip points 104 on the predistribution plate 100 when assembled. FIG. 5 shows a bottom view of the final acid distribution plate 200 which shows the final drip points 204. In FIG. 6, the a bottom view of the final acid distribution plate 200 is shown with one of the inserts 203 removed, which exposes the flow channels 205.

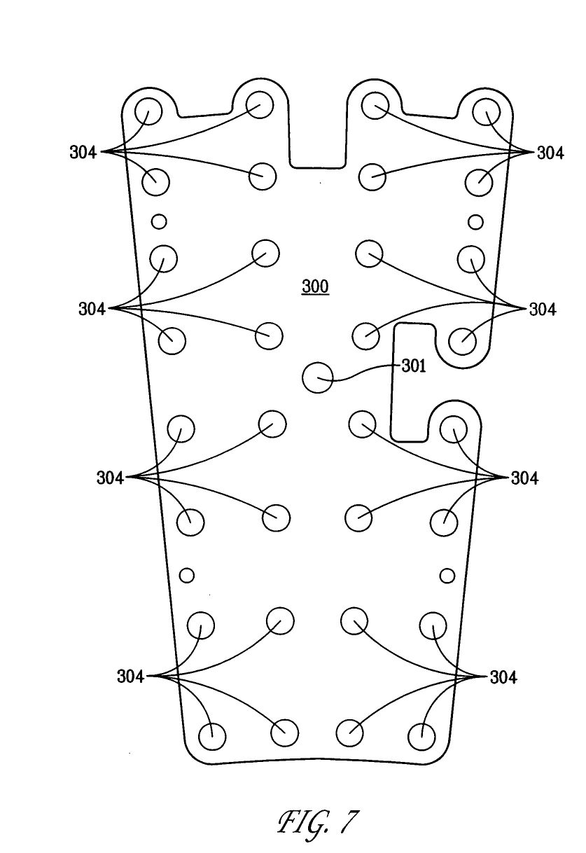

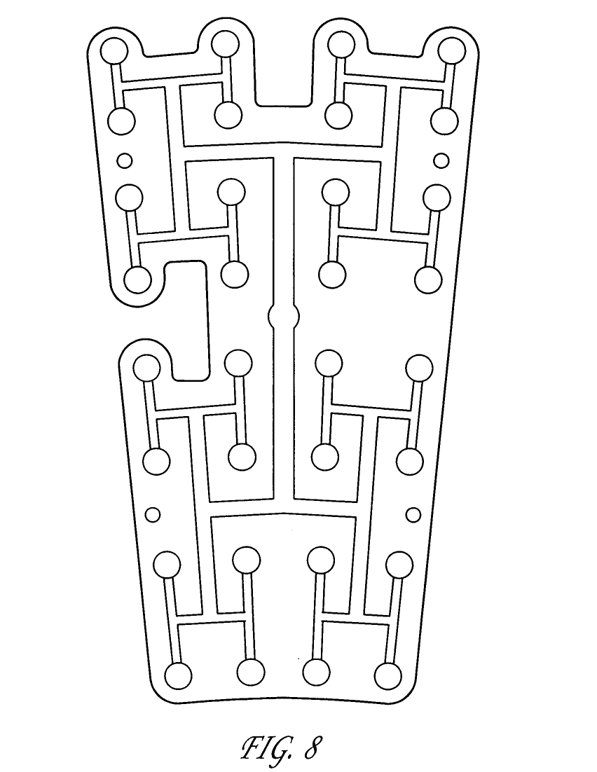

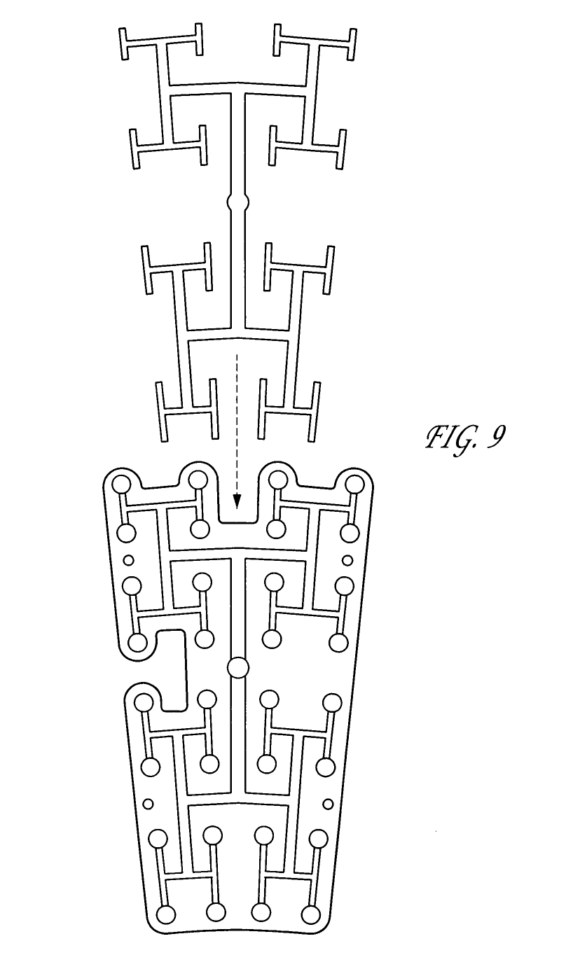

Referring now to FIGS. 7-9 the hydrocarbon distribution plate 300 is depicted. In FIG. 7, a bottom view, the hydrocarbon inlet is shown at 301. There is one hydrocarbon distributor plate 300 per each final acid distribution plate 200. The final outlets or drip points for the acid/hydrocarbon mixture are shown at 304. FIG. 8 depicts the hydrocarbon distribution plate 300 from above with the acid inlets 306 which match up to each of the final acid drip points 204. Insert 303 covers flow channels 305, which can be seen in FIG. 9. The hydrocarbon enters through inlet 301 and mixes with the acid in flow channels 305 and the mixture exits through final drip points 304 into reactor.

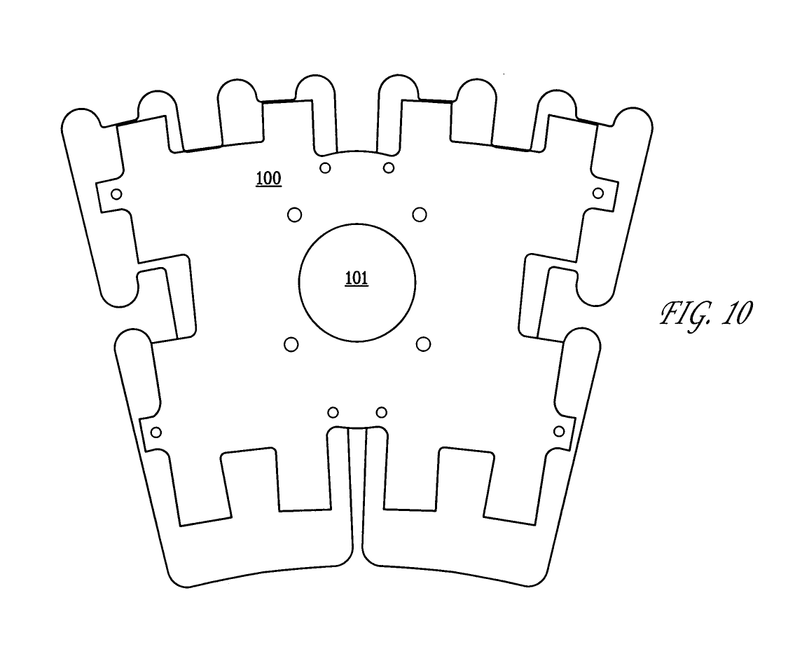

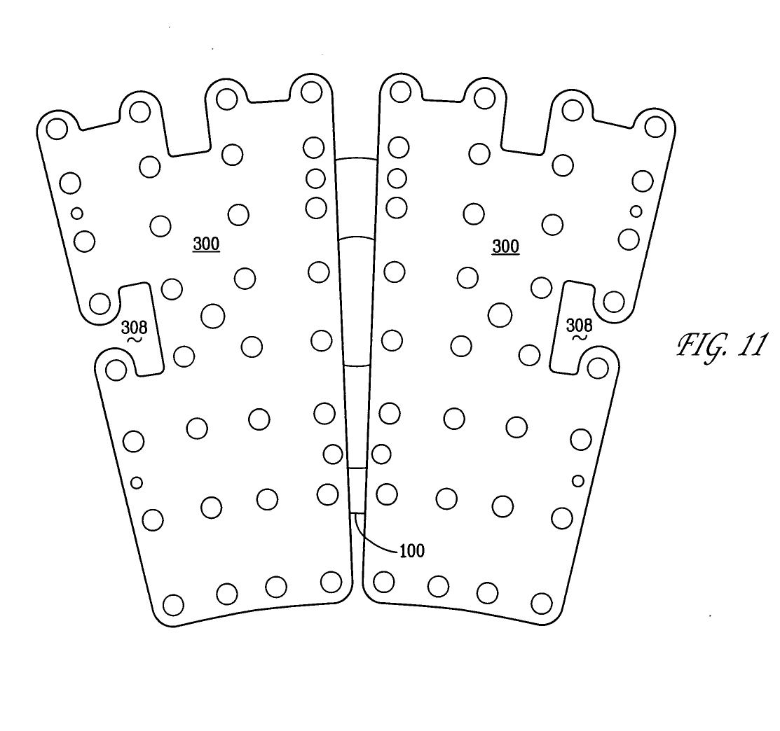

FIGS. 10 and 11 depict a top and bottom view of the assembled plates respectively. Spaces 308 on either side of the assembled plates are for the hydrocarbon inlet piping. The stack shown represents one section of the outer circumference of a vessel having circular cross section of 14.5 ft.

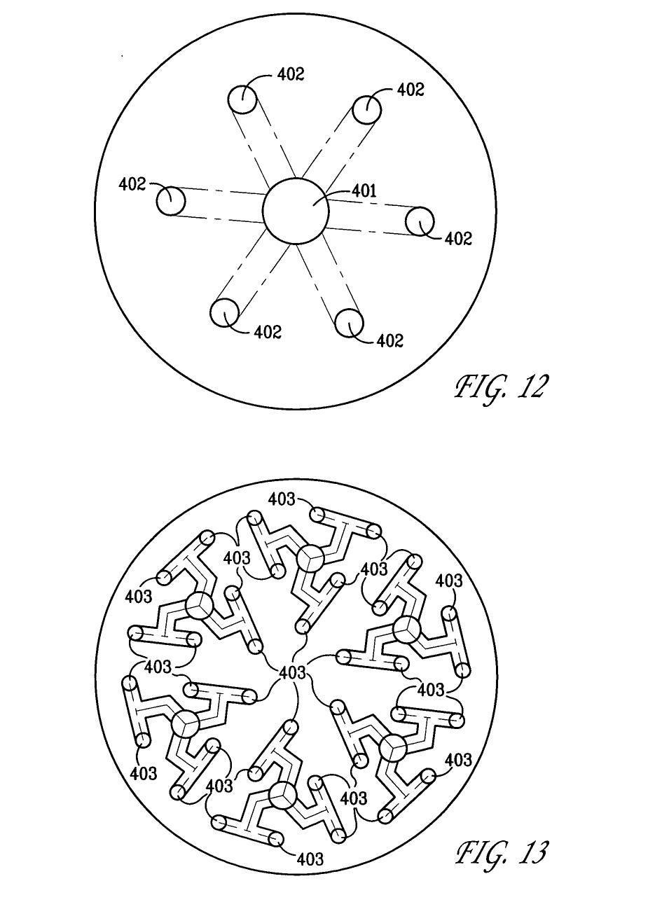

Referring now to FIGS. 12 and 13, the inlet piping for the acid and hydrocarbon is shown. The inlet piping includes a single down spout 401 for each phase which branches into six down spouts 402, each of which branches into six more outlets 403. These outlets are connected to the acid inlet or hydrocarbon inlet on the plate assembly. The inlet is thus fractally branched. The meaning of term “fractally” in this context is “having an equal flow path”. Each branch is a fractal or division. Also, in this context a “fractality” is the point of division.

The problem to get the hydrocarbon inlet piping to the hydrocarbon inlets 301 without the piping interfering with final outlet drip pattern is solved by bring the hydrocarbon inlet piping in overhead along with the acid inlet piping. The hydrocarbon inlet piping, after splitting into 6 overhead pipes, is then passed through spaces 308 at the edge of the plate assemble section and connected to the inlet without passing near or through final drip points 304.

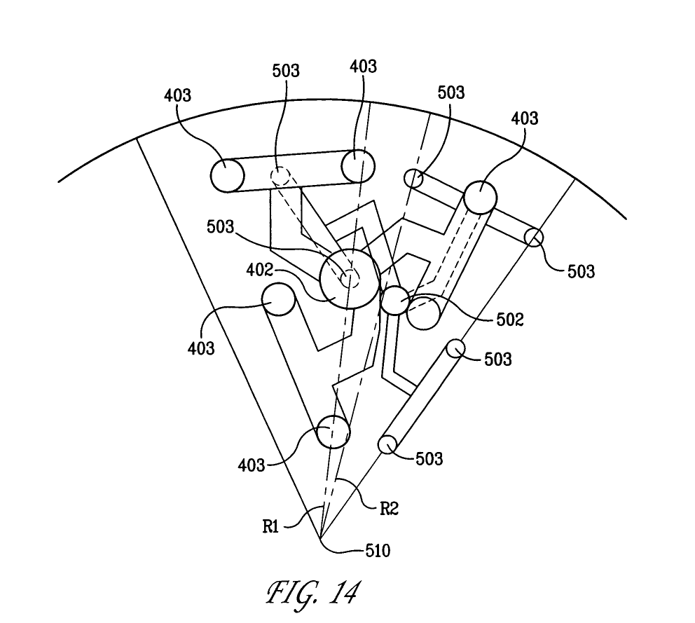

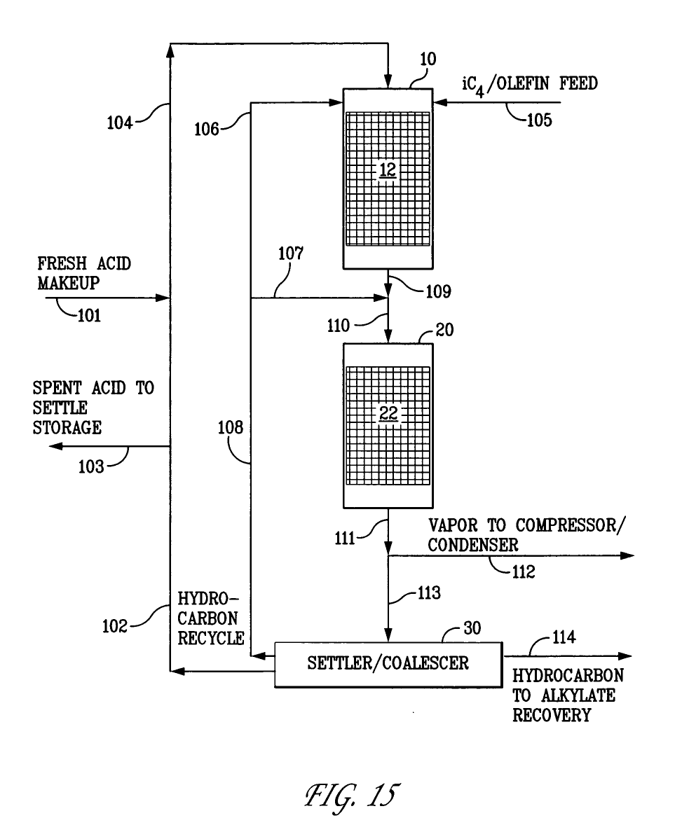

Referring now to FIG. 14, the penultimate down spout 401 of the acid is located central to a wedge 501 of six plate assemblies on a first radius R1. To place the final six downward pipes, or final fractality, of the hydrocarbon inlet pipes 503 over the spaces 308, the radius R2 on which the penultimate down spouts 502 are located, must be rotated around a central axis 510 1/18 of 2π radians (20°) from that of the radius R1, on which the penultimate acid down spouts 402 are located for this particular configuration. As can be seen from FIG. 14, each of the final hydrocarbon down spouts 503 are located on the center of an edge of a plate assembly which corresponds to the location of the spaces 308.

Although three sets of plates are used to illustrate the invention, the first two plates only provide for acid distribution. Only one is used for hydrocarbon distribution. One plate could have been used for the acid distribution. It is contemplated that two plates is the lowest number of plates for mixing two different liquids and many plates in some applications.

### EXAMPLE

To prove the benefits of the utilization of a vaporization section in the overall process, a pilot unit was wherein a single reactor was configured to have a first mixing/reaction zone and a vaporization zone. The pilot unit was operated in the region of “transition” or “pulse” flow in the vaporization zone. The experiment was run as follows:

a) the unit was configured as a downflow reactor;

b) a single packed section was used with a total of 28 1′ tall by 3″ diameter bales being loaded into a combined mixing zone (first reaction zone) and vaporization zone;

c) the packing utilized provided for an acid continuous phase in the mixing and vaporization zones and allowed for a hydrocarbon continuous phase upon exit of the vaporization zone and entry into the coalescer;

d) liquids, recycle acid and hydrocarbon were introduced into the mixing zone;

e) only one recycle hydrocarbon stream was utilized, with that going to the top of the mixing zone;

f) pressure was controlled such that only the bottom 4′ of bales contained vapor;

g) feed of isobutane and olefins containing n-butane was added to recycle hydrocarbon stream at the top of the reactor;

h) a compressor was utilized to remove the heat of reaction with the condensed liquids on the discharge side of the compressor being returned and pumped back to the top of the mixing zone;

i) a packed coalescer was utilized to separate acid drops for a hydrocarbon continuous stream exiting the bottom of the vaporization zone—the hydrocarbon residence time in the coalescer was held to about 2 minutes or less;

j) a portion of the hydrocarbon liquid from the coalescer was sent to a distillation column for product recovery and recovery of isobutane overhead with the overhead isobutane being recycled back to the reactor and mixed with the feed olefins prior to entry into the mixing zone.

The operating conditions are provided in Table I with the feed olefin composition and the resulting alkylate product are provided in Tables II and III respectively.

TABLE I
RUN CONDITIONS

| | |
|---|---|
| Overall mass balanced error, % | −2.5 |
| Average Reactor Temp., F. | 35 |
| Olefin Feed, lb/hr | 18.3 |
| iC₄/Olefin, lb/lb | 6 |
| Pressure drop, psi | 57.3 |
| True alkylate flow rate, lb/hr | 27.3 |
| Acid, wt % | 93.44 |
| Water, wt % | 1.85 |
| Height packing, ft. | 28 |

TABLE II
OLEFIN FEED COMPOSITION

| Component | wt % |
|---|---|
| 1-butene | 17.3 |
| n-butane | 37.3 |
| trans-2-butene | 30.0 |
| 2,2 dimethyl propane | 0.1 |
| methyl cyclo pentane | 0.1 |
| cis-2-butene | 15.0 |
| C₅'s | 0.1 |

TABLE III
ALKYLATE PRODUCT COMPOSITION

| Component | wt % |
|---|---|
| iso-pentane | 3.46 |
| 2,3,dimethyl butane | 3.29 |
| 2 methyl pentane | 0.61 |
| 3 methyl pentane | 0.34 |
| 2,4 dimethyl pentane | 2.19 |
| 2,2,3 trimethy butane | 0.19 |
| cyclohexane | 0.07 |
| 2 methyl hexane | 0.10 |
| 2,3 dimethyl pentane | 1.24 |
| 2,2,4 trimethyl pentane | 29.64 |
| 2,5 dimethyl hexane | 2.92 |
| 2,2,3 trimethyl pentane | 0.00 |
| 2,4 dimethyl hexane | 4.02 |
| 2,3,4 trimethyl pentane | 18.31 |
| 2,3,3 trimethyl pentane | 19.46 |
| 2,3 dimethyl hexane | 2.56 |
| 2,2,5 trimethyl hexane | 2.09 |
| 2,3,4 trimethyl hexane | 0.37 |
| 2,2 dimethyl heptane | 0.00 |
| 2,4 dimethyl heptane | 0.02 |
| 2,6 dimethyl heptane | 0.04 |
| 2,2,4 trimethyl heptane | 0.30 |
| 3,3,5 trimethyl heptane | 0.15 |
| 2,3,6 trimethyl heptane | 0.11 |
| 2,3,5 trimethyl heptane | 0.05 |
| trimethyl heptane | 0.26 |
| 2,2,6 trimethyl octane | 0.88 |
| C₈'s | 0.60 |
| C₉'s | 0.40 |
| C₁₀'s | 0.00 |
| C₁₁'s | 0.02 |
| C₁₂'s | 6.10 |
| C₁₃'s | 0.06 |
| C₁₄'s | 0.05 |
| C₁₅'s | 0.00 |
| C₁₆'s | 0.00 |

The invention claimed is:

1. A process for producing alkylate using sulfuric acid catalyst comprising (a) introducing a hydrocarbon component consisting essentially of an olefin, an olefin precursor or mixture thereof and an isoalkane to a downflow reaction zone containing a disperser, (b) incorporating a vaporization zone within a vessel containing said reaction zone or in a separate vessel which also contains a disperser, and (c) operating the vaporization zone at the bubble point of said hydrocarbon component to allow vaporization of the hydrocarbon by adjusting the flow ratios of hydrocarbon/acid/vapor within said vaporization zone to operate at or near the pulse flow regime at is outlet.

2. A process for producing alkylate using sulfuric acid catalyst comprising feeding a hydrocarbon component consisting essentially of an olefin, an olefin precursor or mixture thereof and an isoalkane to a downflow reaction zone containing a disperser, contacting said olefin, an olefin precursor or mixture thereof and said isoalkane in the presence of liquid sulfuric acid catalyst and react under conditions of temperature and pressure whereby the heat of reaction is at the bubble point of the hydrocarbon component thereby producing vapor, said vapor inducing near pulse flow or pulse flow at or near an outlet to produce a reaction product, and feeding the reaction product to a vaporization zone containing a disperser under conditions to induce near pulse flow or pulse flow at or near an outlet of the vaporization zone wherein a pressure drop across the disperser causes partial vaporization of the hydrocarbon component of said reaction product, quenching of the heat of reaction and cooling of an unvaporized portion of said reaction product.

3. The process according to claim 2 wherein said hydrocarbon component and said liquid sulfuric acid are fractally fed to said reaction zone.

4. A process for the alkylation of an olefin with and alkane in the presence of a liquid acid catalyst comprising the steps of: fractally feeding a liquid acid catalyst through a fractal distributor to distribute the liquid acid catalyst evenly; fractally feeding hydrocarbon comprising an isoalkane and an olefin through said fractal distributor to distribute the hydrocarbon evenly to produce a reaction mixture thereof with said acid catalyst; reacting said isoalkane and said olefin to produce an alkylate; recovering a reaction product comprising said reaction mixture and said alkylate; and separating said reaction product into a hydrocarbon phase and an aqueous phase.

5. The process according to claim 4 wherein said phases are recovered separately

6. A process for the alkylation of an olefin with and alkane in the presence of a liquid acid catalyst comprising the steps of:
(a) feeding to a reaction zone having an inlet and an outlet and containing a disperser, a liquid acid catalyst and a hydrocarbon comprising isoalkane and an olefin;
said disperser intimately contacting the liquid acid catalyst, the isoalkane and the olefin to react a portion of the isoalkane with the olefin to produce a reaction mixture containing liquid acid catalyst, hydrocarbon comprising unreacted isoalkane, unreacted olefin and alkylate product;
(b) feeding the reaction mixture to a vaporization zone having an inlet and an outlet containing and a disperser under conditions to vaporize portion of the hydrocarbon to produce a vapor and cool the reaction mixture whereby the vapor induces a near pulse flow regime at the vaporization zone outlet to produce a stable and tight emulsion;
(c) withdrawing from the vaporization zone a vapor phase containing unreacted isoalkane, unreacted olefin and alkylate and a liquid phase containing liquid acid catalyst and alkylate product; and
(d) separating the liquid acid catalyst from the alkylate product.

7. The process according to claim 6 wherein the liquid acid catalyst is sulfuric acid.

8. The process according to claim 7 wherein the temperature in the reaction zone is between 15 and 70° F., the pressure drop across the reaction zone is between 0.5-10 psi/ft of packing height, the disperser void fraction is between 0.8 and 0.99, the acid concentration entering the reaction zone is greater than 30 vol %, the acid concentration maintained in the reaction zone is greater than 30 vol % and the temperature rise across the reaction zone is less than 5° F.

9. The process according to claim 8 wherein the tightness of the emulsion as measured by its density after settling for 30 seconds is between 1.2 and 1.7 g/cc.

10. The process according to claim 9 wherein the tightness of the emulsion as measured by its density after settling for 30 seconds is between 1.3 and 1.45 g/cc.
